(19) United States
(12) Patent Application Publication
(10) Pub. No.: US 2025/0213260 A1
(43) Pub. Date: Jul. 3, 2025

(54) VASCULAR DEVICE

(71) Applicant: Clearstream Technologies Limited, Enniscorthy, County Wexford (IE)

(72) Inventors: Sean Wall, Enniscorthy, County Wexford (IE); Pat Byrne, Enniscorthy, County Wexford (IE); Derek Doyle, Enniscorthy, County Wexford (IE)

(21) Appl. No.: 18/852,073

(22) PCT Filed: Mar. 31, 2022
(86) PCT No.: PCT/EP2022/058570

§ 371 Date: Sep. 27, 2024

(51) Int. Cl. A61B 17/221; A61B 17/22

(52) U.S. Cl. CPC A61B 17/221; A61B 2017/22034; A61B 2017/22051; A61B 2017/22079; A61B 2017/2212

(57) ABSTRACT

The present disclosure is directed to a vascular device comprising a catheter and a structure as well as a movable macerator. The structure is provided at a distal part of the catheter and comprises a disengaging portion configured to disengage vascular debris from a vessel wall, and a collecting portion configured to collect removed vascular debris. The movable macerator is located at least partially within the structure and/or proximal to the structure and configured to macerate removed vascular debris, so that the device is configured to macerate vascular debris by way of movement of the macerator for facilitating entry into the opening and/or conveyance through the lumen.

### CROSS REFERENCE TO RELATED APPLICATIONS

This application is a U.S. national phase of International Application No. PCT/EP2022/058570, filed Mar. 31, 2022, which is incorporated herein by reference in its entirety.

### TECHNICAL FIELD

The present disclosure refers to vascular devices, in particular thrombectomy or atherectomy devices.

### BACKGROUND

Thrombectomy devices for treating thrombosis in a patient are known. For example, a thrombectomy device is received within (inside) a vessel and includes a coring portion for coring and separating a portion of a thrombus from the vessel wall, and a mesh structure for capturing thrombi. Thrombi are, once disengaged from the vessel wall, captured by a mesh structure. Mesh structures may include different pore sizes that facilitate the extrusion of portions of the thrombus from the vessel wall.

However, mechanisms like this may provide insufficient results. Further improvement as to extraction and retraction of the removed thrombi is desired.

### SUMMARY

A device facilitating macerating clot/tissue/plaque/calcifications etc. (in the following referred to as vascular debris in general) may help to remove vascular debris from a vessel wall and/or break up vascular debris after removal and, hence, to facilitate efficiency of the procedure.

The present disclosure is directed to a vascular device comprising a macerator. The device comprises a catheter, optionally having a lumen for conveying vascular debris, the lumen optionally having an opening for receiving the vascular debris, the catheter defining a longitudinal axis, the device further comprising a structure provided at a distal end of the catheter, the structure comprising a disengaging portion configured to disengage vascular debris from a vessel wall, and a collecting portion, optionally distal to the disengaging portion, configured to collect vascular debris. Further, the device comprises a movable macerator located at least partially within/inside the structure and/or proximal to the structure and/or distal to the opening. The macerator is configured to macerate vascular debris, in particular whilst removing, i.e. while and/or after vascular debris is removed from the vessel wall. The device may be configured to macerate vascular debris by way of movement of the macerator, e.g. for swiftly breaking up vascular debris in situ (which supports continued disengagement from the vessel wall), for facilitating entry into the opening and/or conveyance of the vascular debris through the lumen.

In some embodiments, the macerator moves such that movement of the macerator agitates and/or breaks up vascular debris, while the vascular debris is within or proximal to the structure. Vascular debris may still adhere to the vessel wall when being agitated. Specifically, before the vascular debris enters the opening into the catheter and is conveyed and/or aspirated through the lumen, the vascular debris may be broken up/macerated, after and/or while the vascular debris has been removed from the vessel wall by way of the disengaging portion. Breaking up the vascular debris and, thus, reducing the size of the vascular debris may allow for faster and more reliable transport of the vascular debris along the lumen of the catheter and, hence, may increase the efficiency of the procedure.

Optionally, the device may be a venous thrombectomy device for removal of venous vascular debris from a vein. The device may be configured for treatment of deep vein thrombosis or pulmonary embolism. The device may be a varicose vein ablation device.

A (venous) thrombus may be blood debris at a vessel wall (of a vein). Vascular debris may refer to acute and/or chronic debris and may include calcifications.

The disengaging portion may be configured for disengaging vascular debris from the vessel wall and may be referred to as a coring element. Generally, the disengaging portion may be configured with a leading edge, allowing for slicing of the vascular debris from the vessel wall. It may be configured to be in contact with the vessel wall by having substantially circumferential parts which engage the vessel wall upon movement of the structure/disengaging portion in the axial direction, specifically upon retraction in a proximal direction.

The structure may represent a single entity combining the disengaging portion and the collecting portion. Multiple parts, including, for example, the disengaging portion and the collecting portions, may be regarded as forming the structure.

The collecting portion may be configured to collect thrombi and emboli, so as to avoid embolism. The collecting portion may also be referred to as a basket and may represent a capturing region, in particular for capturing loose debris.

Generally, the disengaging portion may be proximal to the collecting portion. However, embodiments are conceivable where the disengaging portion is distal to the collecting portion if, for example, additional closing means for closing the opposite side of the vessel are being used.

It may be conceivable that the macerator is within the structure, specifically within the disengaging portion and/or collecting portion of the structure. In one embodiment, the macerator may be provided in the disengaging portion of the structure. The structure may serve as a protection, as the vessel wall, specifically the endothelial layer, is prevented from dissection by way of the macerator. For example, expansion of the macerator may be constrained by the structure, i.e. the diameter of the disengaging portion and/or the collecting portion, to avoid endothelial layer damage.

The catheter defines a longitudinal axis in the length direction of the catheter. The longitudinal axis may, during use, deviate from a straight line, due to tortuous anatomy along which the catheter is being moved.

Specifically, a rotatable macerator may be expandable in the radial direction perpendicular to the axis. Evidently, the effect of the rotational movement is achieved by way of rotation around the longitudinal axis. For a macerator to have a radial extension, it is conceivable that the macerator is compressible and expandable, so as to allow for delivery through the catheter.

Optionally, the structure is expansible and compressible. If the structure, specifically the disengaging portion and/or the collecting portion thereof, is expansible and compressible, the structure may be used also in small vessel applications and may be versatile as to delivery. At the same time, when in the expanded configuration, cross-sectional coverage may be enhanced. Specifically, the disengaging portion may be expansible toward the vessel wall, as this supports contact with the vessel wall and, hence, full-cross-sectional separation of vascular debris from the vessel wall.

In an optional configuration, the macerator is configured to be actively movable. For example, the movement can be based on ultrasound and/or rotation, such as by way of a rotating bar or wire. Hence, the macerator may represent an active (mechanical) macerator.

Optionally, the macerator is rotatable around the longitudinal axis. Rotation of the macerator may allow for more efficient breaking up of vascular debris. Specifically, in some embodiments, rotation of the macerator may initiate rotation of the vascular debris within the structure, which supports breaking up of the vascular debris and reducing the size of debris portions.

Additionally or alternatively, the macerator may be axially movable. The axis corresponds to the longitudinal axis of the catheter at that location. Axial movement may be initiated by an ultrasound drive, for example, which allows for reciprocating movement of the macerator with ultrasound frequency. Such movement initiates back-and-forth movement of the vascular debris in the axial direction and, thus, supports breaking up of the vascular debris, optionally by way of cavitation. This may be particularly beneficial for hard vascular debris, including calcifications.

Optionally, the macerator is deployable from a compressed state to a deployed state. Put differently, the macerator may be expandable, optionally in the radial direction substantially perpendicular to the longitudinal axis. This may facilitate full-cross-sectional vascular debris engagement and maceration. In some embodiments, this allows for improved handling and the application in smaller vessels, as the full volume, specifically cross-section, of the macerator is only needed when the macerator is in the deployed and expanded state, whilst the macerator is compressed during delivery.

In an optional embodiment, the macerator comprises a hook and/or an angled bar, and/or has a ring and/or a star shape. These configurations allow for engagement with vascular debris and, after the engaged vascular debris has been moved and/or while vascular debris is being removed, for efficient separation and breaking up of the vascular debris. Ideally, the macerator extends in the radial direction and is rotatable. This helps to engage a larger area of the vascular debris and, hence, to agitate a larger amount of vascular debris, leading to more efficient breaking-up of vascular debris.

In a further embodiment, the structure may comprise struts. The struts may represent a stent-like configuration. The structure may be referred to as a stent-triever structure. The struts may form a fenestrated or braided mesh structure. This represents an efficient configuration of a structure that allows for collecting vascular debris and/or disengaging from a vessel wall and compression and expansion of the structure as needed. The struts may be interconnected so as to form the structure, specifically a unitary structure. The struts may comprise nitinol. The struts may be made of a memory shape alloy or polymer, such as nitinol and/or a nitinol alloy.

The disengaging portion may comprise a first region of struts and the collecting portion may comprise a second region of struts, wherein the first region of struts differs from the second region of struts. Specifically, the struts of the first region and of the second region may be interconnected so as to form a continuous structure.

It is conceivable that the struts of the disengaging portion are configured for disengaging vascular debris from the vessel wall. For example, the disengaging portion may have leading edges. As such, the struts may have sharp edges for cutting vascular debris from the vessel wall. Struts in the second region comprised in the collecting portion may have no leading edges but may have smooth edges/surfaces.

An additional or alternative difference between the first region of struts and the second region of struts may be that the first region of struts is less dense than the second region of struts as struts in the first region of struts are further spaced apart from each other than struts in the second region of struts. In other words, the second region may be denser than the first region, as the second region serves for collection of vascular debris, whilst the first region serves to cut the vascular debris and to receive the vascular debris inside the disengaging portion of the structure. Through the spaces between the struts of the first region, the vascular debris can enter and pass through the struts.

The distance between the struts in the first and second regions may be determined by the angle of the struts relative to each other. As such, the angle between crossing struts may be smaller in the second region than in the first region.

In an embodiment, the collecting portion may comprise a basket and/or has at least partially a substantially cylindrical shape. The basket and/or the cylindrical configuration of the collecting portion conforms to the vessel walls and is configured to at least partially engage the vessel walls after removal of the vascular debris from the vessel walls by way of the disengaging portion, which may particularly be located proximal to the collecting portion.

The disengaging portion may at least partially have a conical shape. Specifically, the disengaging portion may be tapered, wherein the tapered end is proximal, and the wider end is distal.

The mesh formed by the struts in the first and second region may, in a cross-sectional view, have a circular configuration. This may allow for enhanced collection of vascular debris and/or disengaging of vascular debris from the vessel wall irrespective of the angular position of the structure.

Optionally, the structure is tethered to the distal end of the catheter. Tethering may be an efficient and simple mechanism to provide the structure to the distal end of the catheter. If the structure is tethered to the catheter, the macerator may be proximal to the structure, i.e. between the catheter and the structure. Of course, other configurations are conceivable.

Optionally, the catheter comprises, in addition to the lumen for conveying vascular debris, a separate lumen for receiving and/or handling the structure and/or the macerator. Accordingly, the structure and/or the macerator can be delivered smoothly, without impacting the conveyance/aspiration of vascular debris.

In particular, the lumen for conveying the vascular debris and the lumen for receiving and/or handling the structure and/or macerator may be arranged coaxially or offset to each other. An offset lumen may be employed to facilitate the independent translation of the structure and/or macerator. The structure may be connected to either a central, co-axial lumen or an eccentric lumen whereas the macerator is connected to the alternate lumen or lumens. The structure may be tethered to the proximal end/hub of the catheter via a single or multiple wires through the lumen(s), whereas the macerating element may be connected through a single lumen through the shaft to an energy source at the proximal end of the catheter.

The locations for the lumens may be selected and, depending on the macerator, for example, an appropriate arrangement such as a coaxial or an offset configuration can be selected. In some embodiments, this allows to optimize the space inside the catheter.

Optionally, a membrane is provided in and/or over the collecting portion and/or distal to the collecting portion. Such membrane may, in some embodiments, add protection as small embolic particles can be captured. The membrane may be configured to at least partially occlude a vessel during the procedure. The membrane may be a metallic or polymer impermeable or porous membrane.

Further embodiments will be evident from the below description of the drawings.

### BRIEF DESCRIPTION OF THE DRAWINGS

FIG. 1 shows a longitudinal cross-sectional view of a thrombectomy device.

FIG. 2a shows a configuration of a macerator.

FIG. 2b shows another configuration of a macerator, in a transversal cross-sectional view.

FIG. 2c shows another configuration of a macerator.

FIG. 2d shows another configuration of a macerator.

FIG. 3a shows a cross-section of a catheter having an offset arrangement of the lumens, in a transversal cross-sectional view.

FIG. 3b shows a cross-section of a catheter having a coaxial configuration of the lumens, in a transversal cross-sectional view.

FIG. 4 shows a side view of a structure having a membrane.

FIG. 5 shows a side view of a thrombectomy device having a tethered configuration.

### DETAILED DESCRIPTION OF EMBODIMENTS

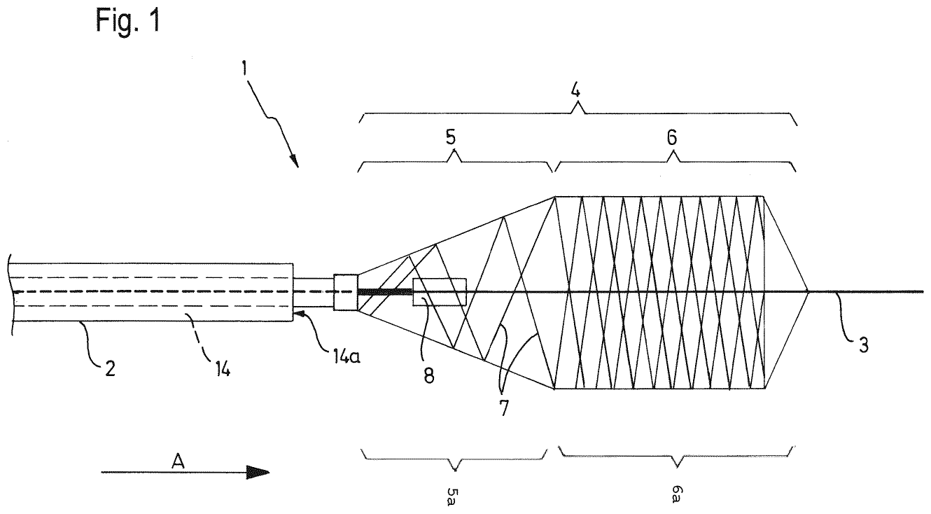

FIG. 1 shows a thrombectomy, or vascular, device 1 according to the disclosure. However, other vascular devices such as atherectomy devices are conceivable.

The thrombectomy device 1 comprises a catheter 2, wherein the catheter 2 defines a longitudinal axis A along the length direction of the catheter 2. The device 1 comprises a structure 4 at a distal part/end of the catheter 2. As used herein, term “proximal” means closer to or in the direction of an origin of an element, such as the vascular or thrombectomy device 1. The origin of the vascular or thrombectomy device 1 may be a handle or other user-manipulated portion of the vascular or thrombectomy device 1, such as of the catheter 2. The term “distal” means further from the origin, or handle, of the vascular or thrombectomy device 1. Put another way, the term “distal” means closer to or in the direction of a tip of the vascular or thrombectomy device 1. The distal direction corresponds to the arrow defining the axial direction in FIG. 1, i.e. pointing to the right side. The structure 4 comprises a disengaging portion 5 which is proximal to a collecting portion 6. The disengaging portion 5 is configured to disengage vascular debris from a vessel wall (not shown in the drawings) when the structure 4 is inside a vessel, i.e. when the vessel walls surround the structure 4. The collecting portion 6 is configured to collect vascular debris, i.e. vascular debris after it has been disengaged and removed from the vessel wall by the disengaging portion 5.

The collecting portion 6 may comprise a basket portion having a substantially cylindrical shape. The disengaging portion 5 may at least partially comprise a conical shape. The cone widens in the distal direction (i.e. to the right side in FIG. 1) and may at its distalmost end correspond to the width of the collecting portion 6.

Struts 7 form the disengaging portion 5 and the collecting portion 6. The struts 7 are continuous and differ as to the angle in the disengaging portion 5, on the one hand, and the collecting portion 6, on the other hand. In other words, the angle of the struts with respect to each other is a first angle in the disengaging portion 5, and the angle of the struts with respect to each other is a second angle (different to the first angle) in the collecting portion 6.

The struts 7 may comprise a first region of struts 5a (in the disengaging portion 5) and a second region of struts 6a (in the collecting portion 6a), see FIG. 1.

FIG. 1 also shows a guidewire 3 along which the catheter 2 and the structure 4 are guided.

The macerator 8 according to one or more embodiments of the present disclosure is indicated in FIG. 1. The macerator 8 is located within the disengaging portion 5 of the structure 4. The macerator 8 can macerate vascular debris (not shown) after having been removed by the disengaging portion 5 from the vessel wall (not shown), or while being removed by the disengaging portion 5 from the vessel wall, by moving the macerator 8 and, hence, the vascular debris within the structure 4. This movement facilitates breaking-up of vascular debris.

The catheter 2 comprises a lumen 14 having an opening 14a at its distal end. The opening 14a is for receiving vascular debris (such as by way of aspiration) and the lumen 14 conveys the vascular debris to a proximal part of the catheter 2.

When the structure 4 is provided distal to the catheter 2 and, hence, distal to the opening 14a, the vascular debris has been macerated once it is received in and/or aspirated through the opening 14a, as vascular debris is aspirated in the proximal direction (the left direction in FIG. 1). During a procedure, the structure 4 may be retracted, i.e. moved in the proximal direction.

With continued reference to FIG. 1, the struts 7 of the disengaging portion 5 have a leading edge. This means that the edges of the struts 7 of the disengaging portion 5 pointing to the proximal direction (i.e. toward the catheter 2 and/or away from the collecting portion 6) are sharp, so as to facilitate cutting/scraping off of vascular debris from the vessel walls.

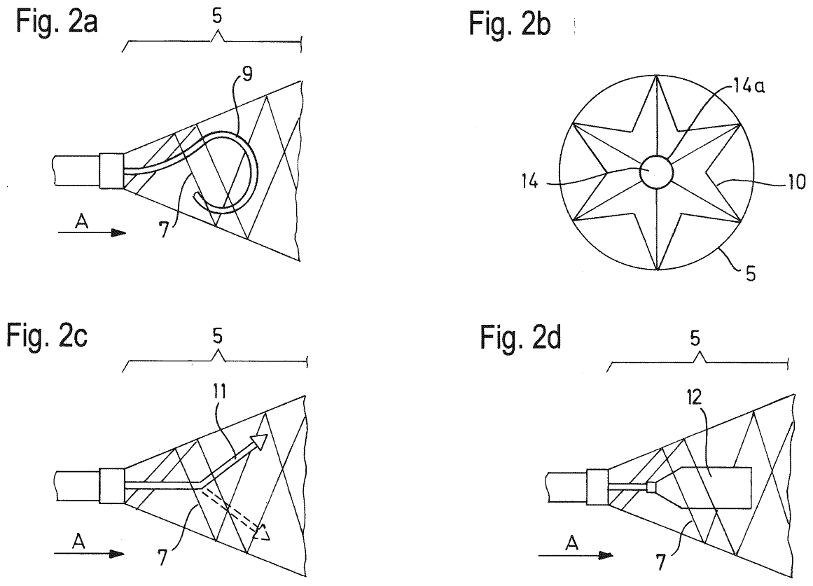

FIGS. 2a to 2d disclose various configurations of a macerator 8 shown in FIG. 1. The macerator 8 is an active means in that it is actively movable. The active element may be single use battery operated, but may alternatively be capital based. In the embodiments of FIGS. 2a to 2c, the macerator 8 is rotatable around the longitudinal direction A.

As shown in FIG. 2a, the macerator 8 may comprise a hook 9 or may have a hook-shape. FIG. 2a shows the hook 9 within the conical disengaging portion 5. The hook 9 is rotatable around the axis A.

In an alternative or additional configuration, the macerator 8 may have an expansible element 10, which is rotatable. The expansible element 10 has, in the cross-section along the transverse direction shown in FIG. 2b a star-shape and is received within the disengaging portion 5 of the structure 4. The opening 14a may be centrally integrated for aspirating vascular debris while cutting.

FIG. 2c shows a macerator 8 having an angled bar 11. Specifically, the bar 11 is angled with respect to the longitudinal direction A. As indicated in FIG. 2c, upon rotation of the angled bar 11 about the longitudinal axis A, rotation of 180° leads to the dashed position.

Alternatively or additionally, as shown in FIG. 2d, the macerator 8 may have an ultrasound element 12. The ultrasound element may initiate a movement along the axial direction A.

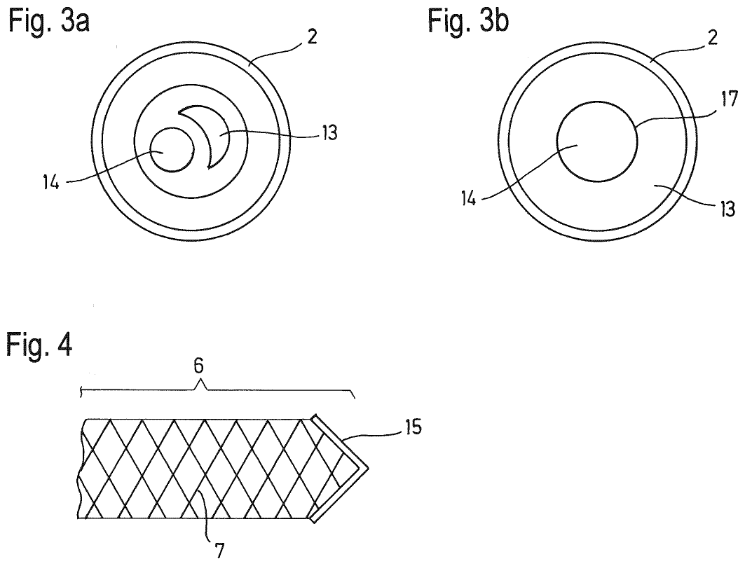

FIGS. 3a and 3b show cross-sections in the transverse direction of the catheter 2. In FIG. 3a, the catheter 2 comprises, in addition to the lumen 14 for receiving vascular debris, a lumen 13 for receiving and/or connecting to the macerator 8 and/or the structure 4. Such configuration of the lumens 14 and 13 is referred to as offset configuration, as the lumens 13 and 14 are offset relative to each other. For example, the cross-section of the lumen 13 may be crescent-shaped.

Alternatively, in FIG. 3b, the lumens 13 and 14 may be arranged coaxially, meaning that, for example, the lumen 13 for receiving or connecting to the macerator and the structure is outside the lumen 14 for receiving the vascular debris and the catheter walls. An inner sheath 17 separates the outer lumen 13 from the inner lumen 14.

FIG. 4 shows a membrane 15, which is provided at the distal end of the collecting portion 6. Specifically, the membrane 15 may be provided in connection with all embodiments, optionally at the distal end of the collecting portion 6. It is also conceivable that the membrane 15 is provided further distal to the collecting portion 6 on a separate entity (not shown in FIG. 4). The membrane 15 is denser than the mesh formed by the interconnected struts 7 of the collecting portion 6, so that smaller embolic particles can be collected, which might have passed through the collecting portion 6, specifically the voids between the struts 7.

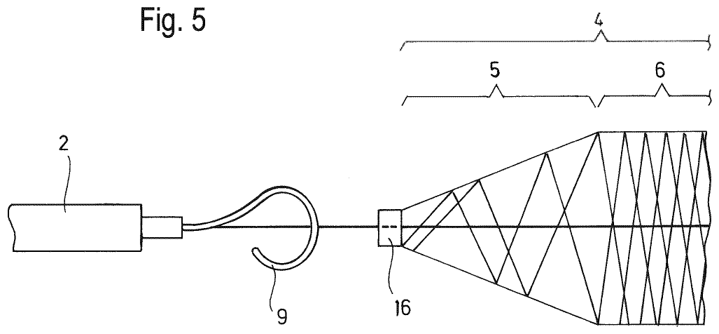

FIG. 5 shows an embodiment in which the structure 4 is tethered to the distal part of the catheter 2. Specifically, a coupler 16 is provided for connecting the structure 4 to the catheter 2. As shown in FIG. 5, a macerator 8 exemplified as the hook 9 may, specifically when the structure 4 is tethered to the catheter 2, be proximal to the structure 4. In other terms, the macerator 8 (here the hook 9) is outside and proximal of the structure 4, i.e. between the distal end of the catheter 2 and the proximal end of the disengaging portion 5. All of the macerators shown above may be used with a tethered structure.

A method according to the disclosure may be defined by the following steps:
1.) The structure 4 and the macerator 8 are distally advanced from the catheter 2 and/or radially expanded, in the case of the structure 4 to contact a vessel wall. 2.) The structure 4 is retracted in the proximal direction and/or compressed in the radial direction against the vessel wall, i.e. to engage the vascular debris. In doing so, the sharpened leading edges of the disengaging portion 5 remove debris from the vascular wall. Simultaneously, the collecting portion 6, having the denser struts 7 at sharper angles relative to one another, catches the removed debris and maintains the debris in the structure 4 (the membrane 15 may assist). 3.) As the structure is further moved proximally and/or collapsed radially, the removed debris is transported to the macerator 8, which is axially/rotationally moved to break the debris into small elements. 4.) Moreover, as the structure 4 is moved proximally and/or collapsed radially, the debris broken up the macerator is moved to a distal end of a lumen 14 of the catheter 2 where it is aspirated through an opening 14a and the lumen 14 of the catheter 2 in the proximal direction.

### List of Reference Signs

1 Vascular (thrombectomy) device
2 Catheter
3 Guidewire
4 Structure
5 Disengaging portion
6 Collecting portion
7 Strut
8 Macerator
9 Hook
10 Expansible element
11 Angled bar
12 Ultrasound element
13 Lumen for receiving and/or connecting to macerator/structure
14 Lumen for receiving vascular debris
14a Opening
15 Membrane
16 Coupler
17 Inner sheath
A Axial direction

1. A vascular device comprising a catheter having a lumen for conveying vascular debris, the lumen having an opening for receiving the vascular debris, the catheter defining a longitudinal axis, the device further comprising:
a structure provided at a distal part of the catheter, the structure comprising:
a disengaging portion configured to disengage vascular debris from a vessel wall, and
a collecting portion configured to collect vascular debris, and
a moveable macerator located at least partially within the structure or proximal to the structure, the moveable macerator located distal to the opening and configured to macerate vascular debris.

2. The vascular device of claim 1, wherein the structure is expansible and compressible.

3. The vascular device of claim 1, wherein the macerator is rotatable around the longitudinal axis.

4. The vascular device of claim 1, wherein the macerator is axially moveable in the direction of the longitudinal axis.

5. The vascular device of claim 1, wherein the macerator (8) is expandable in a radial direction substantially perpendicular to the longitudinal axis (A).

6. The vascular device of claim 1, wherein the macerator (8) comprises at least one of a hook and angled bar, and/or has at least one of a ring-shape and a star-shape.

7. The vascular device of claim 1, wherein the structure (4) comprises struts (7), optionally representing a stent-like configuration.

8. The vascular device of claim 7, wherein the disengaging portion comprises a first region of struts and the collecting portion comprises a second region of struts, wherein the first region of struts differs from the second region of struts.

9. The vascular device of claim 8, wherein the first region of struts is less dense than the second region of struts and/or struts in the first region of struts are further spaced apart from each other than struts in the second region of struts.

10. The vascular device of claim 8, wherein the first region of struts comprises struts having leading edges.

11. The vascular device of claim 1, wherein the collecting portion comprises a basket and/or has at least partially a substantially cylindrical shape.

12. The vascular device of claim 1, wherein the disengaging portion at least partially comprises a conical shape.

13. The vascular device of claim 1, wherein the structure is tethered to the distal end of the catheter.

14. The vascular device of claim 1, wherein the macerator is configured to be actively moveable by way of ultrasound-drive and/or rotation of a driving bar/wire.

15. The vascular device of claim 1, wherein the catheter comprises, in addition to the lumen for conveying vascular debris, a lumen for receiving and/or handling the structure and/or the macerator.

16. The vascular device of claim 15, wherein the lumen for receiving the vascular debris and the lumen for receiving and/or handling the structure/macerator are arranged coaxially or offset to each other.

17. The vascular device of claim 1, wherein a membrane is provided in and/or over the collecting portion and/or distal to the collecting portion.
